(19) United States
(12) Patent Application Publication
(10) Pub. No.: US 2009/0186374 A1
(43) Pub. Date: Jul. 23, 2009

(54) APPARATUS FOR MEASURING EFFECT OF TEST COMPOUNDS ON BIOLOGICAL OBJECTS

(75) Inventors: Alex Okun, San Diego, CA (US); Gregory Kaler, San Diego, CA (US); Ilya Okun, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Etogen Scientific, San Diego, CA (US)

(21) Appl. No.: 12/035,205

(22) Filed: Feb. 21, 2008

Related U.S. Application Data
(63) Continuation-in-part of application No. 12/018,677, filed on Jan. 23, 2008, now abandoned.

(51) Int. Cl. C12Q 1/02; C12M 1/34

(52) U.S. Cl. 435/29; 435/287.3

(57) ABSTRACT

An apparatus and method for real-time measurement of an effect of different concentrations of a test compound or series of test compounds on living cells, in which a flow of cell suspension is combined with a flow of the test compound and a cellular response of the living cells is repeatedly measured by a detector along a length of a detection zone where the cell suspension-test compound mixture is situated. The apparatus may be used in automated screening of libraries of compounds, and is capable of real-time variation of concentrations of test compounds and generation of three-dimensional dose/response/time profiles within a short timespan.

### RELATED APPLICATIONS

This application is a continuation-in-part of U.S. application Ser. No. 12/018,677, entitled APPARATUS FOR MEASURING EFFECT OF TEST COMPOUNDS ON BIOLOGICAL OBJECTS, filed Jan. 23, 2008.

### BACKGROUND OF THE INVENTION

1. Field of the Invention

This invention relates generally to an apparatus for the screening and pharmacological profiling of compounds modulating a cellular physiological response. This invention also relates to devices for rapid assessment of the properties of compounds that modulate the activities of cells. More specifically, this invention relates to devices that facilitate detecting, evaluating, and characterizing the ability of compounds to modulate specific receptor or ion channel functions leading to changes in both the intracellular concentrations of cellular metabolites or ions (for example, calcium ions) detectable by means of reporter compounds (for example, calcium-sensitive dyes), or the intracellular concentrations of reporter compounds (for example, trans-membrane electric potential-sensitive dyes), or the optical properties of the cells (for example, cellular fluorescence, luminescence, light scattering).

The compounds investigated may be involved in regulating the activity of signal transduction pathways, cellular responses, cell surface receptors, ion channels, non-selective pores, second messenger pathways, downstream signal transduction pathways, apoptosis, cellular necrosis or any other cellular responses. In some embodiments, this invention relates to methods and apparatus for detecting, evaluating and characterizing the ability and potency of substances to act as agonists or antagonists against receptors and ion channels localized on a cell surface membrane.

2. Description of the Related Art

Biological cells contain receptor molecules located on their external membrane. The function of these receptors is to “sense” the cell environment and supply the cell with an input signal about any changes in the environment. Cellular environments include interaction with neighboring cells. Receptors allow cells to communicate with each other directly (paracrine regulatory system) or indirectly (endocrine regulatory system), thus achieving harmonized response of a tissue, organ, or a whole organism. Having received such a signal from, for example, a neurotransmitter, hormone, chemoattractant or chemorepellant, the surface localized receptors transmit this information about the extracellular environment to the cell through specific intracellular pathways. This information allows the cell to respond in a specific fashion to accommodate these changes. When there is an altered supply of the external signal molecules or an altered activity of the cell surface molecules, the cell response would be abnormal, causing malfunctioning of a tissue or an organ.

In eukaryotic cells, receptor molecules determine the selective response of the cell. Each type of receptor can interact only with a specific set of ligand molecules. For example, adrenergic receptors interact with adrenaline and noradrenaline, cholinergic receptors interact with acetylcholine, serotoninergic receptors interact with 5-hydroxytriptamine, dopaminergic with DOPA and so on. The cells derived from the different tissues invariably express specific sets of tissue receptors. Different types of receptors are connected to different signal transduction pathways. For example, nicotinic cholinergic receptor, upon binding acetylcholine molecule, directly activates sodium channel (Claudio et al., 1987, herein incorporated by reference in its entirety). G-protein coupled receptors activate enzymes of second messenger pathways, for example, adenylate cyclase or phospholipase C with subsequent activation of cAMP or phosphoinositide cascades (Divecha and Itvine, 1995, herein incorporated by reference in its entirety). Receptor tyrosine kinases activate cascade of MEK/MAPK kinases leading to cell differentiation and proliferation (Marshall, 1995 and Herskowitz, 1995, herein incorporated by reference in their entirety). Cytokine receptors activate JAK/STAT cascade which in turn can regulate other pathways as well as activate gene transcription (Hill & Treisman, 1995, herein incorporated by reference in its entirety).

Together with the receptors, the cell surface membrane carries ion pumps, ion transporters and ion channels. These molecular assemblies work in concert to maintain intracellular ion homeostasis. Any changes in the activity of these systems would cause a shift in the intracellular concentrations of ions and consequently to the cell metabolic response.

Ion pumps act to maintain transmembrane ion gradients utilizing ATP as a source of energy. The examples of the ion pumps are: Na⁺/K⁺-ATPase maintaining transmembrane gradient of sodium and potassium ions, Ca²⁺-ATPase maintaining transmembrane gradient of calcium ions and H⁺-ATPase maintaining transmembrane gradient of protons.

Ion transporters use the electrochemical energy of transmembrane gradients of one ion species to maintain gradients of other ion counterpart. For example, the Na⁺/Ca²⁺-exchanger uses the chemical potential of the sodium gradient directed inward to pump out calcium ions against their chemical potential.

Ion channels, upon activation, allow for the ions to move across the cell membrane in accordance with their electrochemical potential. There are two main types of ion channels: voltage operated and ligand-gated. Voltage operated channels are activated to the open state upon changes in transmembrane electric potential. Sodium channels in the neuronal axon or L-type calcium channels in neuromuscular junctions exemplify this kind of channel. Ligand-gated channels are activated to the open state upon binding a certain ligand with the chemoreceptor part of their molecules. The classical example of ligand-gated channels is nicotinic cholinergic receptor which, at the same time, is the sodium channel.

The majority of drugs developed to combat various diseases act by modulating cell receptor functions and/or ion channels to either inhibit or stimulate their activities. Such drugs act as modulators by stimulating either a synergistic or antagonistic affect on endogenously present biological stimuli (ligands).

One of the major characteristics that determine the therapeutic effect of a potential drug is its potency, which also correlates to the concentration of the drug that needs to be given and maintained in a patient blood to positively affect the diseased state. The drug potency, in turn, is determined by its affinity for a corresponding receptor.

There are numerous methods for detecting ligand/receptor interaction. The most conventional are methods where the affinity of a receptor to a substance of interest is measured in radioligand binding assays. In these assays, one measures specific binding of a reference radiolabeled ligand molecule in the presence and in the absence of different concentrations of the compound of interest. The characteristic inhibition parameter of the specific binding of the reference radiolabeled ligand with the compound of interest, IC₅₀, is taken as a measure of the affinity of the receptor to this compound (Weiland & Molinoff, 1981 and Swillens et al., 1995, herein incorporated by reference in their entirety). Recent advances in microchip sensor technology made it possible to measure direct interactions of a receptor molecule with a compound of interest in real time. This method allows for determination of both association and dissociation rate constants with subsequent calculation of the affinity parameter (Fagerstam et al., 1992, herein incorporated by reference in its entirety). While being very precise and convenient, these methods are not effective to distinguish between agonist and antagonist activity of the compound.

The type of biological activity of the compounds, agonist or antagonist, may be determined in the cell based assays. In the methods described in Harpold & Brust, 1995, which is incorporated herein by reference in its entirety, cells cotransfected with a receptor gene and reporter gene construct, are used to provide means for identification of agonist and antagonist potential pharmaceutical compounds. These methods are inconvenient because they require very laborious manipulations with gene transfection procedures, are highly time consuming and use artificially modified cells. Besides, to prove that the agonistic effect of a particular compound is connected to the stimulation of a transfected receptor, several control experiments with a positive and negative control cell lines should be performed as well.

Other prior art methods use natural cells and are based on registering the natural cell responses, such as the rate of metabolic acidification, to the biologically active compounds. The disadvantage of the prior art is low throughput speed, each measurement point taking about three minutes. Another disadvantage of the prior art is the use of cells immobilized on the internal surface of the measuring microflow chamber. This leads to the necessity of using separate silicon sensors, or cover slips, with the cells adherent to them for each concentration point of the agonist or antagonist, for the receptors that undergo desensitization upon binding to the agonist molecule. This results in high variability of the experimental results.

Ionized calcium, unlike other intracellular ion events, e.g. changes in the intracellular concentrations of protons, sodium, magnesium, or potassium, serves as the most common element in different signal transduction pathways of the cells ranging from bacteria to specialized neurons (Clapham, 1995, is incorporated herein by reference in its entirety). There are two major pools which supply signal transduction pathways in the cell with the calcium ions, extracellular space and the endoplasmic reticulum. There are several mechanisms to introduce small bursts of calcium into cytosol for signal transduction.

Both excitable and nonexcitable cells predominantly have two receptor classes on their plasma membrane that control calcium entry into cell cytoplasm, G-protein coupled serpentine receptors (GPCSR) and the receptor tyrosine kinases (RTK). Both GPCSR and RTK receptors activate phospholipase C to convert phosphatidylinositol into inositol(1,4,5)-trisphosphate (InsP₃) and diacylglicerol. InsP₃ acts as an intracellular second messenger and activates specialized receptor that spans the endoplasmic reticular membrane. The activation of this receptor triggers release of calcium ions from the endoplasmic reticulum (Berridge, 1993, is incorporated herein by reference in its entirety). The calcium ions can also enter the cytoplasm of excitable and nonexcitable cell from the extracellular environment through specialized voltage-independent Ca²⁺-selective channels triggered by specific ligands. In nonexcitable cells, hyperpolarization of the plasma cell membrane also enhances entry of calcium ions through passive transmembrane diffusion along the electric potential. For example, opening of potassium channels brings the membrane potential to more negative values inside the cell, thus facilitating Ca²⁺ entry across the plasma membrane. Excitable cells contain voltage-dependent Ca²⁺ channels on their plasma membrane, which, upon membrane depolarization, open for a short period of time and allow inflow of Ca²⁺ from external media into cytoplasm. The endoplasmic reticulum membrane as well as plasma membrane of the excitable cells contains InsP₃ receptors and Ca²⁺-sensitive ryanodine receptors (RyR) releasing Ca²⁺ from intracellular stores upon membrane receptor triggered phospholipase C activation or depolarization-induced short burst of Ca²⁺ entry into cell cytoplasm from extracellular media respectively.

It is well established that G-protein coupled serpentine receptors initiate Ca²⁺ mobilization through the activation of phospholipase C_{β} (Stemweis and Smrcka, 1992, herein incorporated by reference in its entirety) whereas tyrosine kinase receptors activate phospholipase C_{γ}, with subsequent intracellular Ca²⁺ mobilization (Berridge & Irvine, 1989, herein incorporated by reference in its entirety).

There are many plasma membrane G-protein coupled serpentine receptors, tyrosine kinase growth factor receptors and voltage- and ligand-regulated channels known to initiate intracellular Ca²⁺ mobilization.

Ca²⁺ plays an essential role in many functional processes of a cell. For example, Ca²⁺ affects the cell cycle (Means, 1994, herein incorporated by reference in its entirety) and activates specific transcription factors (Sheng et al., 1991, herein incorporated by reference in its entirety). Scores of receptors and ion channels use the Ca²⁺ signal to initiate events as basic as cell motility, contraction, secretion, division etc.

Increases in cytosolic and, consequently, in nuclear concentration of the Ca²⁺ can also be a cell death promoting signal. For example, prolonged increase in free Ca²⁺ activates degradation processes in programmed cell death, apoptosis, activates nucleases that cleave DNA and degrade cell chromatin, promotes DNA digestion by direct stimulation of endonucleases, or indirectly by activation of Ca²⁺-dependent proteases, phosphatases and phospholipases, resulting in a loss of chromatin structural integrity (Nicotera et al., 1994, herein incorporated by reference in its entirety).

A development of intracellular fluorescent calcium indicators (Grynkiewicz et al., 1985, herein incorporated by reference in its entirety) made it possible for intracellular concentration of free calcium to be measured directly in the living cell. Thus the ability to register changes in intracellular calcium concentration provide the means for monitoring effects of different compounds useful in treating various diseases, whose action is thought to be a result of an interaction with membrane receptors and ion channels.

There are multiple cell-based assay where the biological activity of a compound can be determined is based on measuring changes in intracellular ion concentrations upon activation of cell receptors and/or ion channels. Different devices have been developed to detect and characterize such receptor-activated or ion channel-activated trans-membrane fluxes of ions. For example, U.S. Pat. No. 5,355,215, herein incorporated by reference in its entirety, discloses a method and apparatus for quantitative fluorescence measurement, whereby measurement of the fluorescence of a layer of cells, disposed in a well, with supernatant liquid there above, is greatly enhanced in sensitivity by illuminating the cell layer with a beam of light incident thereupon at a first angle and detecting fluorescence emitted by the cells with a detector which views the illuminated cells at a second angle, wherein at least one of the first or second angles is oblique to the cell layer. U.S. Pat. Nos. 6,573,039, 6,727,071, and 6,902,883, incorporated by reference in their entirety, disclose an optical system for determining the distribution, environment, or activity of fluorescently labeled reporter molecules in cells for the purpose of screening large numbers of compounds for specific biological activity. The method comprises providing cells containing fluorescent reporter molecules in an array of locations and scanning numerous cells in each location with a fluorescent microscope, converting the optical information into digital data, and utilizing the digital data to determine the distribution, environment or activity of the fluorescently labeled reporter molecules in the cells. The array of locations may be an industry standard 96-well or 384-well micro titer plate or a micro plate with a micro patterned array of locations of fluorescently labeled cells in each well of the plate. The invention includes apparatus and computerized method for processing, displaying and storing the data.

Though the described devices allow for identification of compounds that affect cell functional and biochemical responses in a high throughput manner, they are only capable of assessing kinetic changes in the developing signal and do not provide concentration-dependent characteristics of the compound interaction with the cells, which are vital for determination of their potency and efficacy. To be able to do so, additional liquid handling equipment is necessary, which makes such systems extremely expensive.

Another device, developed to measure the concentration-dependent effects of the receptor or ion channel ligands, is based on a flow-through technique whereby a compound is diluted in-line to a variable concentration profile and the flow of the formed compound concentration gradient is mixed with the flow of the cells in a mixing chamber. (U.S. Pat. Nos. 5,804,436, 5,919,646, 6,096,509, 6,242,209, 6,280,967, and, 6,379,917, all herein incorporated by reference in their entirety). The combined stream passes from the mixing chamber, through a connecting tube, and into a flow-through cuvette of a reading device. The resulting signal is continuously registered by the reading device while the mixture passes through the flow-through cuvette. The signal represents continuous changes of the cell reaction to variable compound concentration at a given time point, the compound concentration being determined by the flow rate and connecting tubing length. Thus, this apparatus provides dependence of the signal on the compound concentration. A disadvantage of this device is that while it provides continuous signal/concentration profiles, kinetic characteristics of the signaling can only be assessed by repeating measurements of the concentration curve, which can only be accomplished by changing the length of the tubing connecting the mixing chamber with the flow-through cuvette of the reading device. This is a very time consuming procedure, requiring calibration of the system after each change of tubing.

Therefore, one embodiment of this invention provides an inexpensive apparatus capable of measuring concentration and time-dependent changes in cell signaling with respect to both activators and inhibitors of the signal.

This can provide major value for the basic science of cell receptor signaling, as well as drug discovery and drug development, by providing means to thoroughly characterize the pharmacology of the drugs on specific receptors, ion channels and other therapeutic targets.

Another embodiment of this invention provides an apparatus for continuous monitoring of cell response in order to test substances in a concentration and time-dependent fashion.

### SUMMARY OF THE INVENTION

One embodiment includes an apparatus comprising an automated test compound source capable of providing one or more test compounds at a programmably controlled flow rate, an automated living cell or particle source capable of providing one or more cell or particle types at a programmably controlled flow rate, an automated dilution reagent source capable of providing a dilution reagent at a programmably controlled flow rate, a mixing chamber in fluid communication with the test sample source, the cell or particle source, and the dilution reagent source wherein the mixing chamber is adapted to combine a flow of at least one test compound received from the test compound source with a flow of dilution reagent received from the dilution reagent source, to provide a test solution, which is combined with the cells or particles received from the cell or particle source to generate a combined stream; and a detector, comprising i) a detection capillary liquidly coupled with the mixing chamber to receive and hold the length of the combined stream after the flow is stopped and ii) a detector, for measuring changes in properties of the cells, located along the length of the detection capillary. In an aspect of the embodiment, the automated test compound source and the automated dilution reagent source are capable of creating variable flow rates of the test compound and the dilution reagent, respectively. In an aspect of the embodiment, the flow rate of the test compound increases proportionally to the decrease in the flow rate of the dilution buffer to maintain a constant test solution flow rate while increasing the concentration of the test compound along a stream of the test solution.

Another embodiment further comprises an automated standard reagent source capable of providing one or more standard reagents having a known effect on the cells to the mixing chamber at a programmably controlled flow rate, wherein the mixing chamber is adapted to combine a flow of the standard reagent with a flow of the dilution reagent to provide a standard solution that can be combined with cells or particles received from the cell or particle source to generate a combined mixture flow. In one aspect, the flow rate of the standard reagent increases proportionally to the decrease in the flow rate of the dilution buffer to maintain a constant standard solution flow rate while increasing the concentration of the standard reagent along a stream of the reagent solution. In one aspect, the combined mixture flow is comprised of the cells or particles, the test compound, the dilution buffer, and the standard reagent.

In some embodiments, a pump fluidly is coupled with the exit end of the capillary to provide a pull out rate equal to the rate of combined stream entering the capillary from the mixer under positive pressure created by the combined flow rate of fluids into the mixer to eliminate a pressure drop during flow of fluids within the apparatus.

In some embodiments, a wash pump is fluidly coupled with the exit end of the capillary to provide a counter-flow of at least one cleaning solution into the capillary and the mixing chamber and an outlet port on the mixing chamber is coupled with a vacuum source to evacuate the cleaning solution.

In one embodiment, the mixing chamber comprises a cell intake port fluidly coupled with the cell or particle source to receive a stream of cells or particles from the test compound source; at least one test sample intake port to receive the test sample from a nozzle fluidly coupled with the test sample source; a dilution reagent intake port to receive dilution reagent from the dilution reagent source; at least one mixing zone in fluid connection with the cell intake port, the test sample intake port and the dilution reagent intake port; an outlet port coupled with a vacuum source to evacuate a wash solution, and at least one outlet port liquidly coupled with the capillary to provide a combined stream to the capillary.

In one embodiment, the apparatus further comprises a standard reagent intake port to receive standard reagent from the standard reagent source, which is fluidly coupled to the mixing zone.

In some embodiments, the detector is selected from a photometer, a fluorometer, a cytometer, a radiometer.

In some embodiments, the cell types or particle types are located in an array.

One embodiment includes a method for determining a response of living cells to a test sample, comprising providing a flow of a test sample, providing a flow of living cells, combining the flow of test sample with the flow of living cells to form a combined flow of the test sample and the living cells in a mixing zone, directing the combined flow of the test sample and the cells to a capillary, stopping the combined flow when the capillary is filled in, repeatedly detecting and measuring a response of the living cells to the test sample along the capillary length to affect a kinetic analysis of the effect of the test compound on the living cells. In one aspect, combining the flow of test sample with the flow of living cells comprises mixing the test sample and the living cells in a mixing zone. One aspect comprises providing an increasing flow of the test sample, providing a decreasing flow of the dilution reagent, providing a constant flow of the living cells, combining the flow of test sample, dilution reagent and living cells to form a combined flow in the mixing zone, directing the combined flow to a capillary, and stopping the combined flow when the capillary is filled in. In one aspect, the sum of flow rates of the test sample and the dilution reagent is constant, while the concentration of the test sample increases along the combined flow. In one aspect, kinetic responses from different locations in the capillary length represent independent traces from a multiplicity of cells located along the capillary at a constant concentration of the test sample. In another aspect, the kinetic responses from different locations in the capillary length represent independent traces from a multiplicity of cells located along the capillary at variable concentrations of the test sample.

One embodiment comprises determining a concentration-dependent response of the cells to the test sample after combining the test sample with the cells in the mixing zone. In one embodiment, the response of the cells comprises a change in the characteristics of the cells, induced by the test sample. One aspect comprises determining a dose-time-response curve of the test sample on the living cells. One aspect further comprises a retrowash, wherein at least one wash solution is provided through the output extremity of the capillary and vacuumed off through an outlet port in the mixing zone.

One embodiment further comprises providing a flow of a standard reagent, combining the flow of the standard reagent, with the flow of the test sample, the dilution reagent and the living cells to form a combined flow in the mixing zone, directing the combined flow to the capillary, and stopping the combined flow when the capillary is filled in. One aspect comprises providing a constant flow of the standard reagent, providing an increasing flow of the test sample, providing a decreasing flow of the dilution reagent providing a constant flow of living cells;, and combining the flow of standard reagent, with the flow of test sample, dilution reagent and living cells in the mixing zone, directing the combined flow to the capillary, stopping the combined flow when the capillary is filled in. In one aspect, the sum of flow rates of the test sample and the dilution reagent is constant, while the concentration of the test sample increases along the combined flow. In one aspect, the kinetic responses from different locations in the capillary length represent independent traces from a multiplicity of cells located along the capillary at variable concentrations of the test sample in the presence of a constant concentration of the standard reagent.

One embodiment comprises determining a concentration-dependent response of the cells to the test sample in the presence of constant concentration of the standard reagent after combining the test sample, the standard reagent and the cells in the mixing zone. In one aspect, the response of the cells comprises a change in the characteristics of the cells, induced by the standard reagent in the presence of different concentrations of the test sample.

One embodiment comprises determining a dose-time-response curve of the test sample on the living cells in the presence of the standard reagent.

In some embodiments, the test sample is selected from an agonist, an antagonist and an allosteric modulator. In some embodiments, the standard reagent is selected from an agonist, an antagonist and an allosteric modulator.

### BRIEF DESCRIPTION OF THE DRAWINGS

FIG. 1 is a block-diagram of one embodiment of the apparatus of the present invention.

FIG. 2 is a schematic representation of a mixing chamber with one common mixing cavity as implemented in a preferable embodiment of the present invention. FIG. 2 comprises the combination of FIGS. 2a and 2b.

FIG. 3 is a schematic representation of a mixing chamber with two mixing cavities as implemented in a preferable embodiment of present invention. FIG. 3 comprises the combination of FIGS. 3a, 3b, 3c, and 3d.

FIG. 4 is a schematic representation of connections between different modules of the apparatus.

FIGS. 5A-C are flow diagrams of preferred operations of the apparatus.

FIG. 6 represents a diagram describing changes in liquid dispensing rate in time by pumps 110 and 140, which create a test compound concentration gradient.

FIG. 7 represents traces of fluorescence intensity along the optical capillary length when a calibration dye is dispensed into the capillary. FIG. 7 comprises a combination of FIGS. 7a-7c.

FIG. 8 represents traces of fluorescence intensities of FLUO-4 dye from RD cells along the capillary length taken at different time intervals. FIG. 8 comprises a combination of FIGS. 8a-8c.

FIG. 9 represents 3-D image of the cell response to agonist registered in agonist concentration-time coordinates. FIG. 9 comprises a combination of FIGS. 9a-9c.

FIG. 10 represents 3-D image of the cell response to agonist registered in agonist concentration-time coordinates in the presence of antagonist. FIG. 10 comprises a combination of FIGS. 10a-10c.

### DETAILED DESCRIPTION OF THE PREFERRED EMBODIMENT

The present disclosure contemplates for real-time, continuous monitoring and detection of the physiological or pharmacological effect of a test compound on a series of cell types or on a single cell type. In its simplest embodiment, the present disclosure includes a method and apparatus for continuously contacting a single cell suspension, a series of cell suspensions, each of which contains a single cell type included in the series of cell types to be tested, a single cell suspension containing more than one cell type, or a series of cell suspensions containing more than one cell type with a predetermined concentration of at least one potentially active compound, preferably with predetermined concentrations of at least two active compounds. Then, intracellular changes that occur in response to contact between the cells and the active compounds are continuously measured by a detector. In some embodiments, a plurality of cellular responses can be simultaneously evaluated. In some embodiments, one or more cellular responses are evaluated in a single cell.

In other embodiments, a method and apparatus is provided for continuously contacting particles, or a series of particles, each of which contains a particle type included in the series of particles to be tested, a single particle preparation containing more than one type of particle, or a series of particle preparations containing more than one type of particle with a predetermined concentration of at least one molecule to be tested for interaction with the particles. In some embodiments, the particles are contacted with predetermined concentrations of at least two molecules. Then, interactions between the particles and the molecules are continuously measured by a detector. In some embodiments, a plurality of interactions can be simultaneously evaluated. In some embodiments, one or more interactions are evaluated in a particle.

In other embodiments, the present invention allows the monitoring of the physiological effects of test compounds, the pharmacological effects of test compounds, or the biochemical properties of cells to be measured at the level of individual cells or in each of the cell types in a heterogeneous population of cells. In further embodiments, the present invention allows the simultaneous examination of multiple characteristics of each cell or cell type or the examination of heterogeneous or mixed cell populations.

It is contemplated that the present invention will be of major value in high-throughput screening; e.g., in screening a large number of candidate compounds for activity against one or more cell types. It has particular value, for example, in screening synthetic or natural product libraries for active compounds or biochemical characterization.

It is also contemplated that the present invention will be of major value in high-throughput screening of a sample for a plurality of molecules, such as biological molecules. The present invention can be used to screen a sample for the presence of a large number of biological molecules such as polypeptides, receptor ligands, enzymatic substrates, agonists or antagonists of enzymatic or receptor activity, or nucleic acids.

In one preferred embodiment, a test compound, a standard compound, and a cell suspension containing the cell type to be tested or particle preparation, or a member of the series of cell types or series of particles to be tested are continuously mixed together and, after an incubation period, are contacted with a detector that measures one or more cellular responses, one or more interactions between the molecules and the particles, or the concentration in the cells or in the intracellular medium of at least one analyte. The area in which the cell suspension is mixed with either the test compound or the standard compound is referred to as the “mixing zone.” In a preferred embodiment, the detector comprises a device capable of analyzing individual cells, individual cell types in a cell suspension, individual particles in a sample, individual particle types in a sample, individual components in a sample, or individual types of components in a sample. In some embodiments, the detector is capable of evaluating a plurality of cellular responses or a plurality of interactions between particles and molecules simultaneously.

The analyte can be any analyte that is readily detectable by detectors and/or detector/chemistry combinations. Thus, various ion or electrolyte concentrations, colorimetric changes, optical density changes, fluorescence, luminescence, pH, gas production, and the like are all readily adaptable for use in the present method and apparatus.

The detector is capable of measuring the particular desired signal, whatever its origin. An optical detector may be a spectrophotometer, spectrofluorometer, or a luminometer. These devices are well-known in the art and are commercially available. If the detector is a direct ion measuring device, it can, for example, comprise a pH sensor or an ion selective electrode. Sodium, calcium, and potassium detectors are examples of such devices.

In detecting ion or electrolyte changes, calorimetric or fluorescent dyes are one particularly preferred embodiment. For example, calcium ion is detectable by such probes as Fura-2, Indo-1, Fura Red or Quin-2, sodium ions by SBFL, proton ions by BCECF, SNAFL, DM-NERF, magnesium ions by Mag-Fura-2 or Mag-Fura-5, chloride ions by SPO, SPA or MOAE. All these dyes are commercially available, for example, from Molecular Probes, Inc., Oregon.

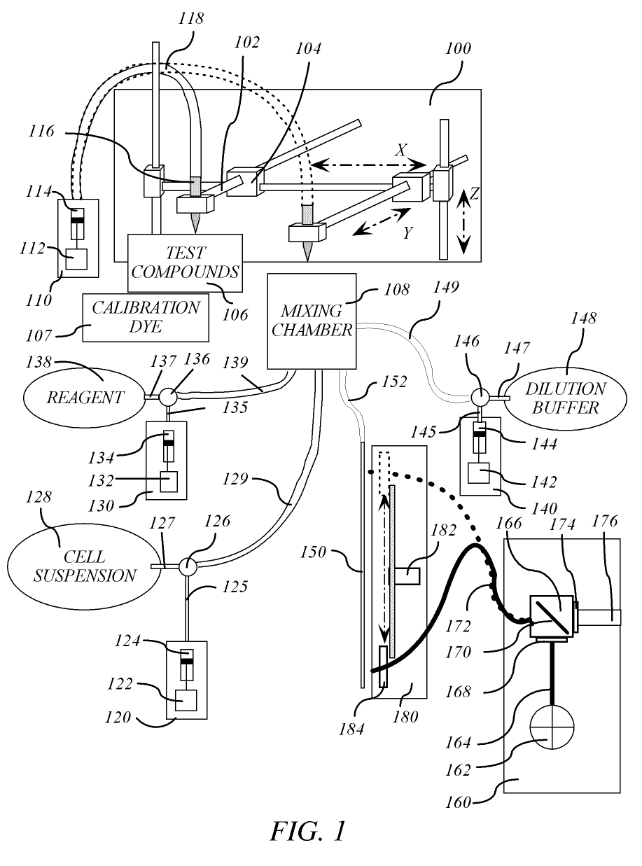

One embodiment is illustrated in FIG. 1. This comprises a sampler 100 for moving a nozzle 116 between positions of a test compound solutions reservoir, 106 or calibration solution reservoir 107, and a mixing chamber 108; an aspirator 110, for aspirating the test compounds or standard solution into nozzle 116 and delivering the test compound or the calibration solution into the mixing chamber 108; a cell delivery structure 120, for delivering a cell suspension into the mixing chamber 108; a reagent delivery system 130, for delivering a reagent with known effect on the cells into the mixing chamber 108; a buffer delivery system 140, for delivering a dilution buffer into the mixing chamber 108; an optically transparent capillary 150, for receiving and holding a combined stream from the mixing chamber 108; a pressure controller 185, for reducing a pressure drop to zero during flow of the liquids in microfluidics channels of the apparatus, and for providing a wash solution into the fluidics system to clean the fluidics channels; an optical excitation and detection system 160, to provide excitation light and to collect an emission light from the capillary; and a detection platform 180, for moving a light head conduit to perform scanning of the signal along the capillary 150.

In an alternative embodiment, parallel fluidics systems can be used to analyze multiple reagents and/or test compounds on one or more cell types. This embodiment comprises of a plurality of samplers 100 and at least one mixing chamber 108 where each sampler moves a nozzle 116 between positions of a test compound solutions reservoir, 106 or calibration solution reservoir 107, and a mixing chamber 108; a plurality of aspirators where each aspirator 110 aspirates a test compound or standard solution into a nozzle 116 and delivers the test compound or the calibration solution into a mixing chamber 108; one or more cell delivery structures 120, wherein a cell delivery structure delivers a cell suspension into a mixing chamber 108; one or more reagent delivery systems 130, where each delivery system delivers a reagent with known effect on at least one cell suspension delivered by a cell delivery structure into a mixing chamber 108; at least one buffer delivery system 140, for delivering a dilution buffer into a mixing chamber 108; an optically transparent capillary 150, for receiving and holding a combined stream from a mixing chamber 108; a pressure controller 185, for reducing a pressure drop to zero during flow of the liquids in microfluidics channels of the apparatus, and for providing a wash solution into the fluidics system to clean the fluidics channels; an optical excitation and detection system 160, to provide excitation light and to collect an emission light from the capillary; and a detection platform 180, for moving a light head conduit to perform scanning of the signal along the capillary 150.

The reservoir for the cells used can be of any shape and form that accommodates access for a nozzle, including, but not limited to, any one of many micro titer plate standards, including, but not limited to, 6-well, 24-well, 96-well, 384-well, and 1536-well plates.

The reservoir for the test compounds used can be of any shape and form that accommodates access for a nozzle, including, but not limited to, any one of many micro titer plate standards, including, but not limited to, 6-well, 24-well, 96-well, 384-well, and 1536-well plates. In some embodiments, parallel fluidics systems can accommodate the simultaneous analysis of multiple test samples and/or cell suspensions by aspirating through multiple nozzles designed to accommodate any one of micro titer plate standards.

The aspirator 110 comprises a syringe 114 driven by a motor 112. The syringe is liquidly coupled with a nozzle 116 through a flexible conduit 118. The nozzle 116 is fastened to a handle 102, which can be moved by the sampler 100 in three dimensions, XYZ, between a test compound solution reservoir 106, to obtain the test compound solution, or a calibration dye reservoir 107, to obtain the calibration dye solution, and a mixing chamber 108, to dispense the said solution into an inlet port 111 of the mixing chamber 108. In one embodiment, the test compound delivery structure, or test compound sample source, provides test compound at a programmably controlled flow rate.

The cell delivery structure 120 comprises a syringe 124 driven by a motor 122. The syringe is liquidly coupled with one port of a first three-way valve 126 through a conduit 125 while a second outlet of the valve is liquidly coupled with a cell suspension reservoir 128 through a conduit 127 and a third outlet of the valve is liquidly coupled with an inlet port 121 of the mixing chamber 108 through a conduit 129. In one embodiment, the cell delivery structure, or automated living cell or particle source, provides cells or particles at a programmably controlled flow rate.

The reagent delivery system 130 comprises a syringe 134 driven by a motor 132. The syringe is liquidly coupled with one port of a second three-way valve 136 through a conduit 135 while a second outlet of the valve is liquidly coupled with a reagent reservoir 138 through a conduit 137 and a third outlet of the valve is liquidly coupled with an inlet port 131 of the mixing chamber 108 through a conduit 139. In one embodiment, the reagent delivery structure, provides reagent at a programmably controlled flow rate.

The buffer delivery system 140 comprises a syringe 144 driven by a motor 142. The syringe is liquidly coupled with one port of a third three-way valve 146 through a conduit 145 while a second outlet of the valve is liquidly coupled with a dilution buffer reservoir 148 through a conduit 147 and a third outlet of the valve is liquidly coupled with an inlet port 141 of the mixing chamber 108 through a conduit 149. In one embodiment, the buffer delivery structure, provides buffer at a programmably controlled flow rate.

The optically transparent capillary 150 is liquidly coupled with an output port 151 of the mixing chamber 108 through a connecting conduit 152, to obtain combined flow of liquids from the mixing chamber 108 provided by the test compound source, the living cell or particle source, the dilution reagent source, and the buffer source.

The optical excitation and detection system 160 comprises a light source 162, to provide an excitation light, a light detector 176, for example, Ultra High Sensitivity CPM Photon Counting Module MP series (PerkinElmer Optoelectronics, USA), to collect emitted light, and a light beam splitter 166, to direct the excitation light onto an entrance of the light guide 172 and direct the emission light onto an entrance of the light detector 176. The light beam splitter 166 comprises a unit 168 comprising a collimation lens with an excitation filter, a unit 174 comprising a collimation lens with an emission filter, and a dichroic filter 170, which reflects the light beam coming from unit 168 with excitation wavelength onto entrance of the light guide 172 and transmits the light beam from the light guide 172 with emission wavelength onto unit 174. The opposite end of the flexible light guide 172 is fastened to a movable scanning head 184 of a motorized detection platform 180, whereby a motor 182 provides movement of the scanning head with the light guide along the capillary 150.

The pressure controller 185 comprises a pressure drop correction pump 190 and a wash pump 187, both pumps being liquidly coupled with valve 186. In a preferred embodiment, the pressure drop correction pump 190 and the wash pump 187 are liquidly coupled with a four-port valve 186. The valve 186 is liquidly coupled with the exit end of the capillary 150 and a waste reservoir 188. The wash pump 187 is liquidly coupled with a reservoir 189 to provide a wash solution into the capillary 150, through the valve 186, and further into the mixing chamber 108 during a wash cycle. The output of the capillary 150 is coupled through the valve 186, either with pump 190 in a run cycle, or with pump 187 in a wash cycle.

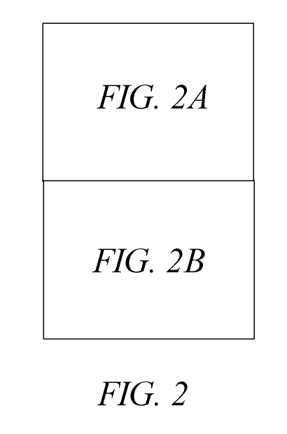

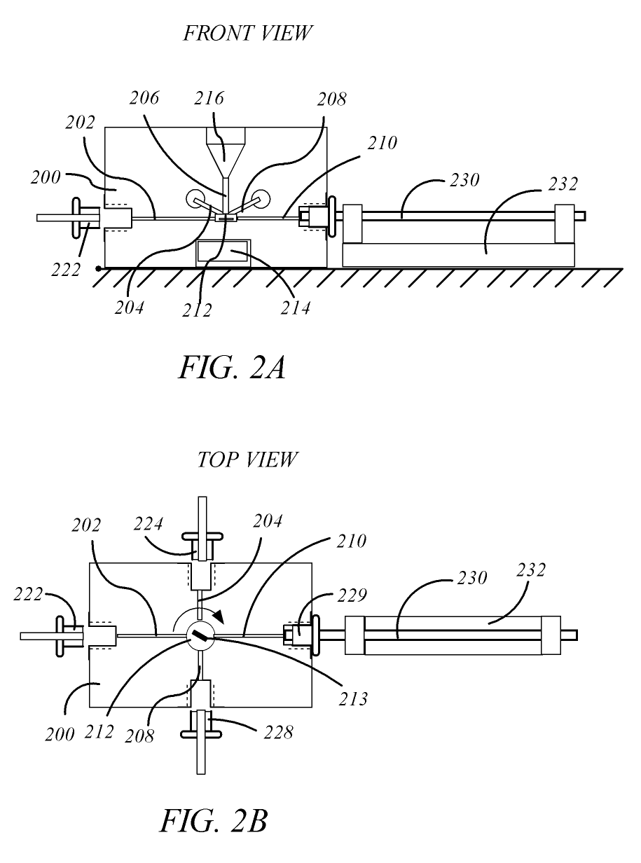

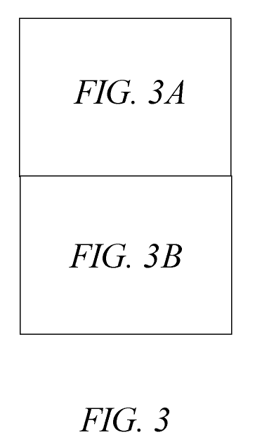

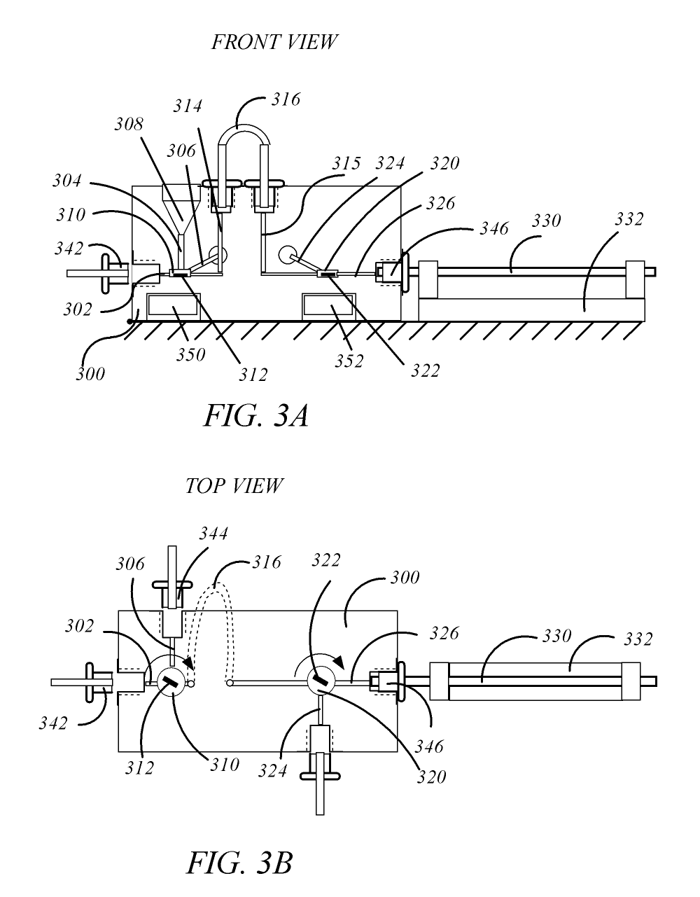

FIG. 2 illustrates one typical embodiment 200 of a mixing chamber 108 of FIG. 1 with one mixing cavity. FIG. 2a represents front view and FIG. 2b top view of the mixing chamber 200. The channel locations and directions are drafted conditionally for a better presentation. It is understood that real positions and directions of the channels may differ from shown. In one embodiment, a mixing chamber 200 is made of chemically inert material, preferably from Teflon (Tetrafluoroethylene) or Delrin (Acetal Copolymer). The mixing chamber comprises intake channels 202, 204, 206, 208, and output channel 210, all of which open into a mixing cavity 212, which may contain a magnetic rod 213 revolving or tumbling when external variable magnetic field generated by a device 214 (for example, MINI 20 from Thermo Electron Corporation, USA) is applied, to efficiently mix liquids flowing through the cavity. Opposite ends of the intake channels 202, 204, and 208 are coupled through connectors 222, 224, and 228 with, referring to FIG. 1, conduits 129, 139, and 149, respectively. The intake channel 206 opens into an injection port 216 to accommodate the nozzle 116 (FIG. 1). When the nozzle is inserted into the injection port, the external edges of the nozzle are sealed against the injection port wall and the liquid flows from the nozzle into the channel 206 and further into the mixing cavity 212. The injection port has an opening 217 coupled with a waste container 240 through channel 218 and conduit 220 with waste reservoir 240, which is under a negative pressure, to evacuate washing solution from the injection port when the device is in a “Wash Mode” as explained in a description for FIG. 3. The output channel 210 opens, through a fitting connector 229, into capillary 230 made of optically transparent material, for example, glass or quartz. The capillary is immovably fixed in a holder 232. Connectors 222, 224, and 226 couple the cavity 212 with pumps 120, 130, and 140 through corresponding conduits 129, 139, and 149, which provide cell suspension, reagent, and dilution buffer, respectively, into the cavity of mixing chamber 108.

FIG. 3 represents another preferable embodiment 300 of the mixing chamber 108 of FIG. 1 with two mixing cavities 310 and 320. FIG. 3a represents front view and FIG. 3b top view of the mixing chamber. As in the case of FIG. 2, it should be understood that real locations and directions of the channels may differ from ones shown in the example. In a preferable embodiment shown in FIGS. 3a and 3b, the intake channels 302, 304, and 306 open into a first mixing cavity 310, which in some embodiments contain a first magnetic stirring rod 312 for efficient mixing of incoming streams. Intake channel 304 couples the first mixing cavity with injection port 308, which accommodates nozzle 116 (FIG. 1) to provide a test compound solution. Opening 318 located in the injection port 308 is coupled with a vacuumed waste reservoir through conduit 319, to evacuate washing solution from the injection port 308 when the device is in the “Wash Mode.” The first mixing cavity 310 is also coupled with pumps 120 and 140 through channels 302 and 306, connectors 334 and 336, and conduits 129 and 149, to provide a cell suspension and dilution buffer from reservoirs 128 and 148, respectively, into the first mixing cavity. The total volume of the channels 314 and 315 and conduit 316 is essentially equivalent to a volume of the capillary 330. Output of the first mixing cavity is coupled with a second mixing cavity 320 through channels 314, 315 and at least one conduit 316. Second mixing cavity 320 is also coupled with pump 130 through channel 324, connector 325, and conduit 139, to provide a stream of a reagent with known effect on the cells, from reservoir 138. In some embodiments, the second mixing cavity 320 also contains a stirring magnetic rod 322, to efficiently mix stream from the first mixing cavity with stream of the reagent. An output of the second mixing cavity is coupled with capillary 330 through channel 326, and fitting connector 338. The capillary is fastened in a holder 332.

Stirring rods 312 and 322 can be actuated in corresponding mixing cavities 310 and 320 by variable magnetic fields created by devices 340 and 342, respectively.

In a “Run Mode” configuration, shown in FIG. 3c, valve 350 liquidly couples an output extremity of capillary 330 with syringe 352 and pump 356 with waste reservoir 258. Simultaneously with pumps (referring to FIG. 1) 110, 120, 130, and 140 pushing into the capillary the test compound solution, cell suspension, reagent, and dilution buffer, respectively, motor 354 moves the syringe plunger down providing a volumetric pull out rate equal to the combined injection rate. The combined pull-push action eliminates pressure drop along the microfluidies channels, stabilizes the flow of the liquids and, hence, improves quality of the experiments. During this cycle, pump 356 is preferably turned off.

In one embodiment, a retrowash is employed. In the retrowash, shown as “Wash Mode” in FIG. 3d, valve 352 switches to an alternative position and couples both the output extremity of capillary 330 with pump 356 and syringe 352 with waste reservoir 360. Motor 354 moves the syringe plunger up, to dispose of the liquid in the syringe and set it up for the next cycle. At the same time, the pump 356 provides wash solution from reservoir 358. In some aspects, additional or alternative wash solutions can be used to serially wash the capillary. These alternative wash solutions can include, but are not limited to, sodium dodecyl sulfate (SDS), ethanol, sodium hypochlorite, DMSO and water. The wash solution is injected into the capillary and further through channels 326, 315, 316, 314, 304, and the first and second mixing cavities into injection port 308, where the wash solution is vacuumed off through conduit 319. It should be understood that valve 350 shown in FIG. 3 may be of any type and configuration known to one of ordinary skill in the art.

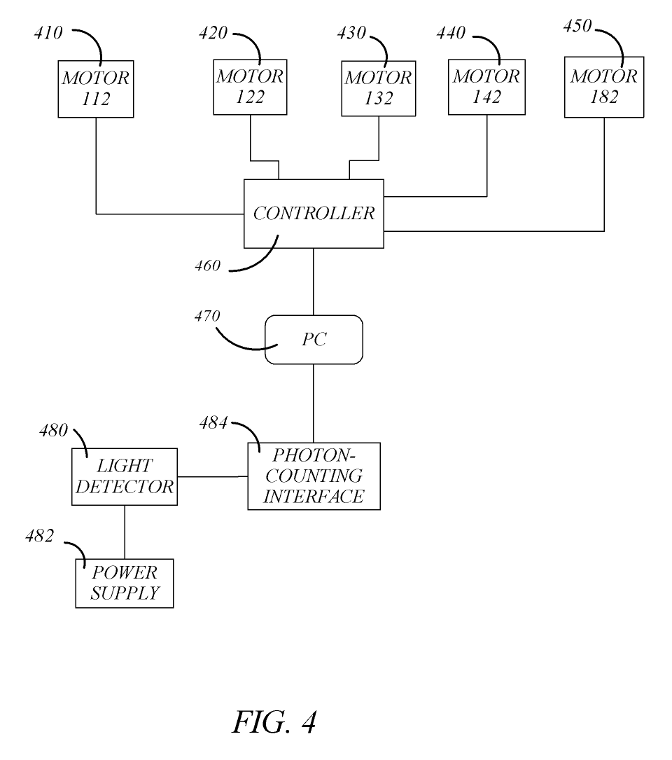

In the preferable embodiment shown in FIG. 4, the motors 410, 420, 430, 440, and 450 of the aspirator, 110, cell delivery structure, 120, reagent delivery system, 130, buffer delivery system, 140, and pressure controller 190, the motor 455 of the detection platform 180, and a motor 465 of the pump 187 (FIG. 1) are connected to and are under control of a controller 460, which provides power input to the motors of the devices and is connected to a computer 470 to receive commands controlling movement of the motors. The computer provides an algorithm in accordance with which the motors move syringes 114, 124, 134, 144, 194 and scanning head 184 (FIG. 1). The light detector 480 (176, referring to FIG. 1) is connected to a power supply 482 and its output is connected to a pulse-counting interface 484, for example, based on LS7366R CMOS counter (LSI Computer Systems, Inc., USA), which transforms photons emitted from the cells into pulse counts per time interval, which is proportional to the intensity of the emitted light. Output of the pulse counting interface is connected to the computer 470 to provide the cell-generated signal information (intensity of emitted light) to the PC software for further calculations.

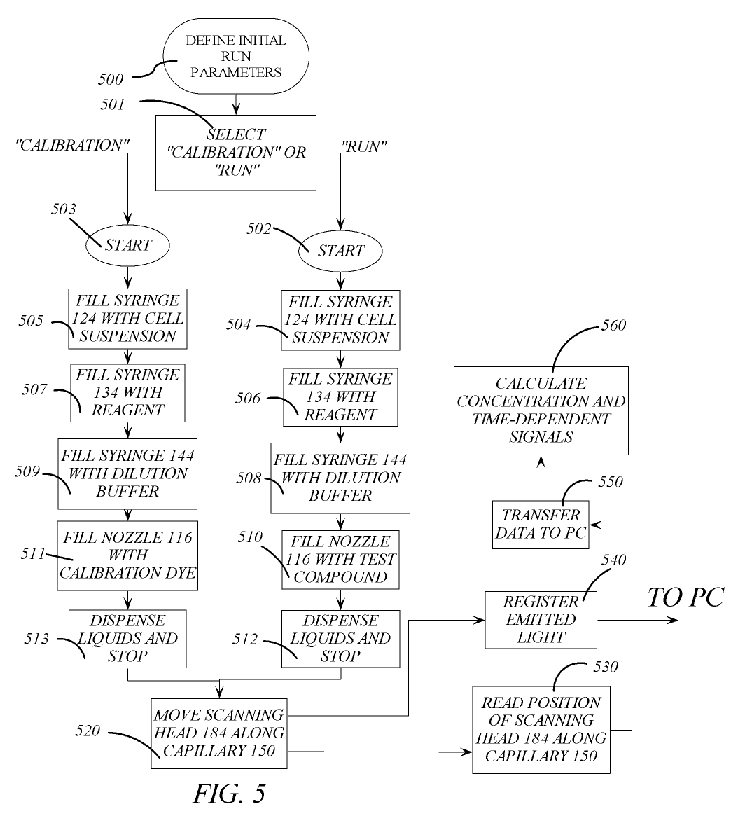

FIG. 5 shows a process flow of one preferable embodiment of the invention. In step 500, the system starts initialization of the apparatus and then in steps 502, 504, 506 fills syringes 124, 134, and 144 (referring to FIG. 1) with cell suspension, reagent, and dilution buffer from respective reservoirs 128, 138, and 148. In step 510, the apparatus finishes the initialization process and prompts an operator to define run parameters for both the fluidics operation (such as positions in the test compound reservoir to be accessed, total volume of the dispensed liquids, rate of dispensing, and steepness of the concentration gradient) and detection operation (such as rate of scanning, integration time and a number of reading cycles). In step 512, the operator selects either a “Calibrate” or “Run” mode. The “Calibrate” and “Run” modes are essentially identical to each other with the only difference that in the “Calibrate” mode, a calibration fluorescent dye is used instead of a test compound to define distribution of the compound concentration along the optical capillary.

When the “Calibrate” mode is selected in step 512, a calibration procedure starts in step 514 (FIG. 5b) whereby the handle 102 of the sampler 100 moves nozzle 116 to the reservoir 107 containing solution of the calibration dye. In step 516, pump 110 aspirates the calibration dye into the nozzle. In step 518, handle 102 of the sampler 100 moves nozzle 116 into the injection port of the mixer chamber 108 and in step 520, pumps 110, 140, 130, and 120 dispense, respectively, the calibration dye, dilution buffer, reagent, and cell suspension in accordance with the run parameters entered in step 510. After a total volume of the liquids predetermined in step 510, is dispensed through mixing chamber 108 into capillary 150, the pumps stop in step 522.

When the “Run” mode is selected in step 512, a run procedure starts in step 515 whereby handle 102 of the sampler 100 moves nozzle 116 to a specified position of reservoir 106 containing solutions of the test compounds. The reservoir for the test compounds can be of any shape and form that accommodates access for nozzle 116, including, but not limited to, any one of many micro titer plate standards, including, but not limited to, 6-well, 24-well, 96-well, 384-well, and 1536-well plates. In step 517, pump 110 aspirates the test compound solution into the nozzle. In step 519, handle 102 of the sampler 100 moves nozzle 116 into the injection port of the mixer chamber 108 and in step 521, pumps 110, 140, 130, and 120 dispense, respectively, the test compound, dilution buffer, reagent, and cell suspension in accordance with the run parameters entered in step 510. The dispensing rates of pumps 120 and 130 are constant thus maintaining constant concentrations of the cells and reagent solution, while dispensing rates of pumps 110 and 140 vary with time in such a way that their combined flow rate is maintained constant thus providing increasing dispensing rate of the compound (or dye) and decreasing dispensing rate of the dilution buffer. This provides a concentration gradient of the compound (or calibration dye) along the capillary with a curvature defined by the operator in step 510.

After a total volume of the liquids predetermined in step 510, is dispensed through mixing chamber 108 into capillary 150, the pumps stop in step 522 and a registration phase begins (step 524). When the pumps and corresponding flow of liquids stop, the cells are evenly distributed in the capillary and, in accordance with the test compound gradient profile, are in contact with the surrounding media containing constant concentration of the reagent (for example, agonist) and variable concentration of the test compound (for example, antagonist). In this case, the effect of different concentrations of the antagonistic test compound on agonist-induced cell response is to be registered. Alternately, the test compound may be an agonist itself and thus, an effect of different concentrations of the agonistic test compound on the cell response will be registered. As cells are not moving when in the capillary, their response to different concentrations of the test compound will be registered by repeating measuring the cell response signal in time along the capillary length.

As the registration phase starts in step 524 detection platform 180 (FIG. 1) moves (in step 526) scanning head 184 along the capillary 150. A light beam from light source 162 passes through a collimator and excitation interference filter 168, to spectrally select wavelength necessary for excitation of intracellular fluorescent indicator dye (for example, 475 nm for intracellular calcium sensitive dye FLUO-4 Invitrogene, Inc.), deflects on the dichroic mirror 170 onto entrance of the light guide 172 and illuminates cells located in the capillary 150 with a narrow light beam. Emitted from the illuminated cells light enters back into the light guide 172, passes through the dichroic mirror 170 and the emission collimator/filter unit 174, to spectrally select a light emitted by the reporter dye in the cells (for example, 550 nm for FLUO-4), and illuminates a light sensitive element of the light detector 176, which registers the signal intensity in step 528. At the same moments as the light intensities along the capillary 150 are registered in step 528, in step 530 detection platform registers corresponding positions of the scanning head. Both the intensities of the signal and corresponding scanning head positions along the capillary are transferred to PC 470 in step 532. Using preliminary performed calibration procedures, which are used to calculate relation between position along the capillary and a concentration of a test compound, the PC then calculates and plots in step 534 dependence of the cell-generated signal as a function of the test compound concentration.

Repetitive scanning along the capillary after the flow is stopped provides measurements of time-dependent changes of the signals generated by the cells in the presence of an agonist and the test compound at different concentration, thus providing series of concentration curves as a function of time. A skilled artisan can easily envision that the test compound may be an agonist and its concentration-dependent stimulation of the cells will be registered.

During the calibration of the instrument in steps following step 512, a signal generated by the calibration dye along the capillary, provides a relationship between the capillary length and the concentration of the substance distributed by combined operations of pumps 110 and 140. This can be achieved by first filling in the capillary with the calibration dye and measuring its fluorescence along the length of the capillary, which gives distribution of the fluorescence intensity for maximal concentration of the dye along the capillary length. Then the capillary is filled with the dye concentration gradient and the second measurement of the fluorescence intensity distribution along the capillary length is taken. By dividing fluorescence intensity obtained in the second step at each point of the capillary length into corresponding fluorescence intensities obtained in first step, one obtains a relationship between a compound concentration conversion coefficient and a position on the capillary length.

Using the compound concentration conversion coefficient, the computer program calculates the test compound concentration profile along the capillary based on the calibration run, and plots dependence of the cell signal as a function of the test compound concentration in real time. It also can plot time-dependent kinetics of the signal generated by the cells at each concentration point of the test compound.

By using known sets of standard agonist and antagonist substances to different receptors, it is possible to screen the compounds against several receptor types and subtypes for specificity and selectivity. When a series of cell types or particle types is to be tested, the process can be repeated for each of the cell types or particle types included in the series of cell types or particle types to be tested in order to evaluate the compounds' activities in a number of cell types or interactions with a number of particle types.

For example, for the endothelin receptor, stimulation of which is indicated by an increase in intracellular concentration of ionized calcium, the following receptor subtype specific antagonists may be used: BQ-123, BQ-788, BQ-153, BQ-485, BMS-182874 PD 151, 242, and the following receptor subtype specific agonists may be used: endothelin-1, endothelin-2, and endothelin-3. Sarafotoxin S6c, IRL 1620, BQ-3020.

For calcium channels, there are sets of channel type specific agonists and antagonists which can be used in a preferred embodiment. For example, agonists of intracellular calcium channels are: Ins(1,4,5)P₃, Ryanodine, Caffeine, Heparine, Perchlorate, and their antagonists are: Decavanadte, Ruthenium Red and high concentrations of Ryanodine.

As discussed above, while the embodiments shown herein include steps for supplying a standard(s), it will be appreciated that these steps need not be present if the analysis being performed does not require the use of a standard compound(s). Instead, the algorithm simply determines whether the cells responded to the test compound(s) or whether the sample contains a molecule.

Cells for use in the apparatus can be selected for the presence of particular known receptors or for their ability to provide predetermined cellular responses to particular stimuli. A large number of such cells are known. For example, to measure the effect of compounds on calcium mobilization induced by different types of receptors, one may wish to use Jurkat T Cells, Platelets, Umbilical Vein, Endothelial Cells, or Chines Hamster Lung Fibroblasts for thrombin receptor; Cerebellar Purkinje Cells, Cortical Astrocytes and Cortical Glial cells for AMPA receptors; Hippocampal Neurons for NMDA Receptor; P-12 cells for Purinergic Receptors; Oligodendrocytes for Platelet-Derived Growth Factor Receptor, Human Neuroblastoma cells and Pituitary Cells for Neuropeptide Y Receptors and protein-tyrosine kinase and protein-tyrosine phosphatase receptors; Human Medulloblastoma cells for Endothelin Receptor; Neutrophils for TNFa Receptor; NG108-15 cells for opioid, bradykainin and ATP; Synovial Fibroblasts for Plasminogen Receptors and so on. If one wishes to measure an intracellular ion concentration, for example, one can preincubate the cells with a dye or other detectable material having sensitivity to concentration of that particular ion. (An actual working example illustrating preparation of cells for detection of calcium ion is set forth in Example 1.)

Alternatively, if one wishes to determine the pattern of natural expression of receptors responsible for Ca²⁺ signaling pathway, then one may use the cells of particular interest and then using a set of agonists known to exert their activity through Ca²⁺ mobilization, to characterize the cells by what type of the receptors are expressed in these particular cells. This set of agonists may consist of acethylcholine, adrenaline, noradrenaline, 5-hydroxitriptamine, DOPA, NMDA, AMPA, Angiotensin II, Bradykinin, Bombesin, Opioid, Endothelin-1 Neuropeptide Y, TNF, PDGF, FGF and others.

The following examples illustrate specific, non-limiting experiments and apparatus operations in accordance with the present invention.

### EXAMPLE 1

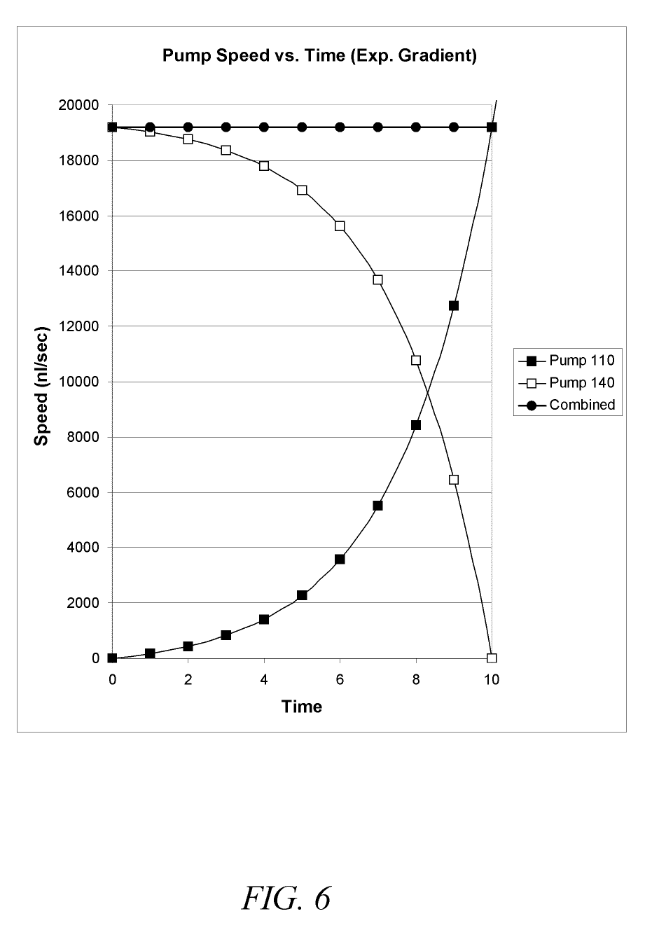

FIG. 6 illustrates time profiles of the pump speeds to distribute a test compound and a dilution buffer during the gradient formation cycle. Pump 140 starts dispensing the dilution buffer from reservoir 148 at a maximal speed with subsequent slowing of the dispensing rate to zero. Simultaneously, pump 110 starts dispensing the test compound from nozzle 116 at zero speed, ramping up the dispensing rate to a maximal value defined by an operator in step 502 (FIG. 5a) as a maximal test compound concentration. The combined flow of test compound and dilution buffer is the test solution. As time progresses, pump 110 increases its pumping rate and pump 140 decreases its pumping rates in a manner where the combined distribution rate of both the test compound and dilution buffer remains constant. Pump 190 works to create a zero pressure drop along the microfluidics system by creating a negative pressure at the end of the capillary 150 in such a way that its rate is equal to the summary rates of all components. For simplicity, FIG. 6 only depicts the two flows provided by pumps 110 and 140 but it is understood that when all pumps are working the rate of pump 190 equals to the combined rate of the other pumps, 110, 120, 130, and 140. The temporal speed profiles for pumps 110 and 140 are calculated by a PC based on the gradient curvature selected by operator in step 502.

### EXAMPLE 2

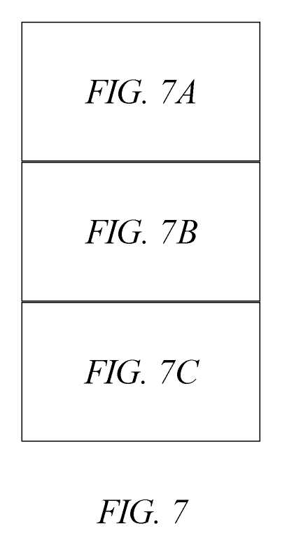

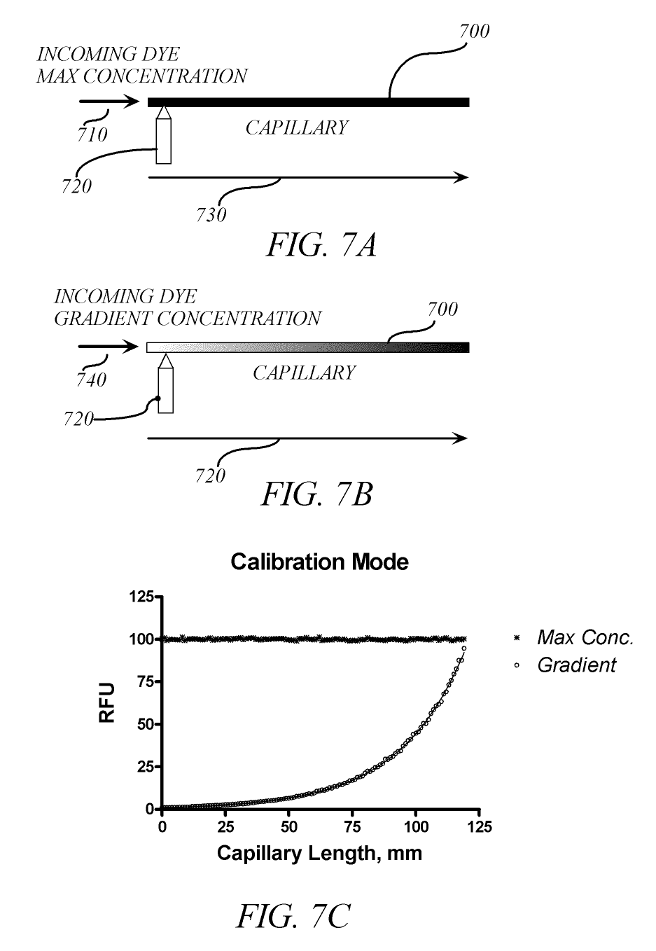

As shown in FIGS. 7a-7c, a calibration procedure comprises of two steps. A maximal concentration of a calibration dye, for example, fluorescein, is dispensed into optical capillary 700 and the flow is stopped. The fluorescent intensity is registered with scanning head 720 by exciting and reading fluorescence of the dye while moving scanning head 184 along the capillary length in the direction shown by arrow 730. The reading of the fluorescence intensity is shown on FIG. 7c denoted as “Max Conc.” The values of the signal are assigned a 100% concentration. In the second step 740 (FIG. 7b), the capillary is filled with a concentration gradient of the dye. The fluorescence signal, which is proportional to the dye concentration as a function of the capillary length, is detected and measured. The fluorescent intensities of the trace, “Gradient” (FIG. 7c), are normalized by the “Max Conc” signal at corresponding points of the capillary length. The obtained traces are fitted to an equation c=A*exp(kl), where c is a concentration coefficient expressed as a function of the capillary length, l, and the coefficients A and k define the shape of the gradient. The experimental fluorescence value curve is shown in FIG. 7c. The calculated relation between concentration coefficient, c, and the capillary length, l, is stored in computer memory and is used to estimate the concentration distribution of the test compound along the capillary length.

### EXAMPLE 3

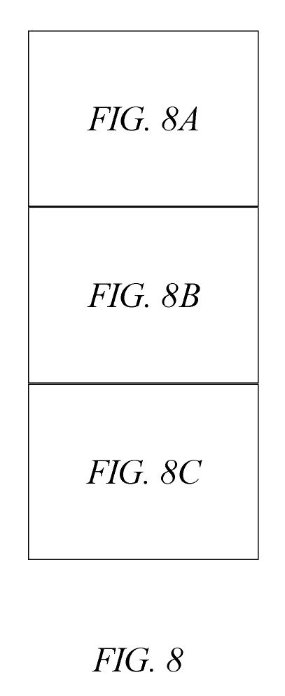

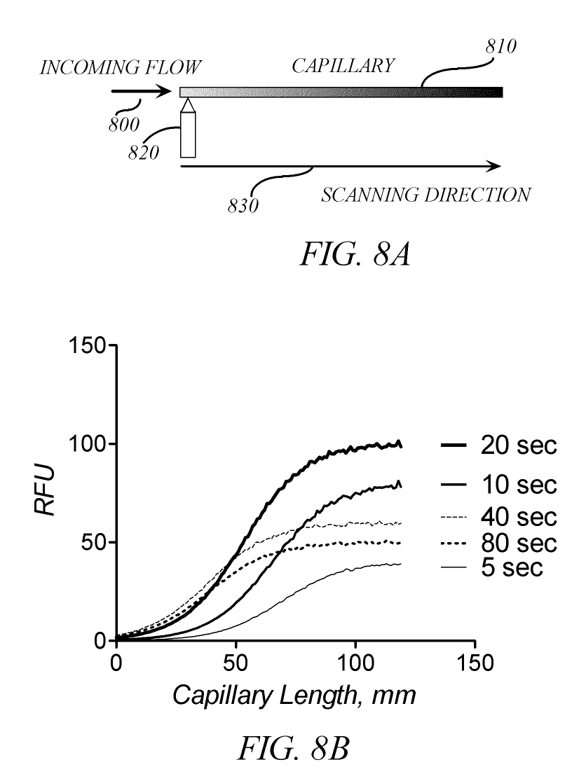

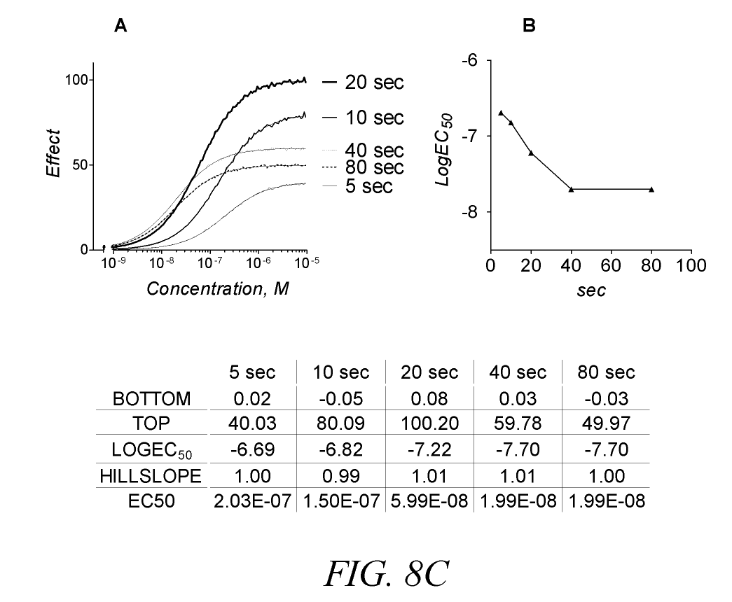

FIG. 8a demonstrates an example of one preferable implementation of the apparatus. RD cells, (former known as TE-671 (ATCC CRL 8805) endogenously expressing muscarine receptors, M3, are loaded with the calcium sensitive dye, Fluo 4. These cells were stimulated with the test compound carbachol, an agonist of acetylcholine receptors. Carbachol is a non-hydrolysable agonist, with an action mechanism similar to that of acetylcholine, which upon interaction with M3 cholinergic receptor, activates phosphatidylinositol phosphate signaling and elevates intracellular calcium concentrations. (Bencherif M, Lukas R J. Ligand binding and functional characterization of muscarinic acetylcholine receptors on the TE671/RD human cell line. J Pharmacol Exp Ther. June 1991;257(3):946-53; Grassi F, Giovannelli A, Fucile S, Mattei E, Eusebi F. Cholinergic responses in cloned human TE671/RD tumour cells. Pflugers Arch. October 1993;425(1-2):117-25; herein incorporated by reference in their entirety). Referring to FIG. 1, the loaded cells were placed into the cell suspension reservoir 128 and upon initiation of the experimental run, the pump 120 aspirated and provided a predetermined volume of the cell suspension into mixing chamber 108 at a predetermined constant flow rate for a predetermined time, as defined by an operator in step 500 (FIG. 5). Simultaneously, pump 130 provided a a buffer solution. The flow rate of the buffer solution was essentially the same as that of cell suspension. At the same time, pumps 110 and 140 provided agonist and dilution buffer, respectively, with the variable in time flow rates in a manner described in Example 1, so that the combined flow rate of the two flows remained constant. The concentration of carbachol was increasing from zero to a maximal concentration as a log function of the capillary length. In the mixing chamber 108, all streams were mixed and the combined stream entered into and filled-in the capillary 150. Accordingly, the flow of cell suspension was continuously mixed with exponentially increasing concentrations of carbachol solution along the length of the combined flow. Referring to FIG. 8a, the formed mixture stream 800 filled the capillary 810 and the flow was stopped. The changes in intracellular concentrations of calcium ions were measured along the capillary 150 as a function of the agonist concentration by registering fluorescence intensity at λₑₘ=516 nm upon excitation with the light beam with excitation wavelength, λₑₓ, 494 nm. The scanning head 820 was activated to move in the direction shown by arrow 830A. Scanning was repeated at different time points (5 sec, 10, sec, 20 sec, 40, see and 80 sec) after the flow was stopped and the fluorescence traces registered at the mentioned time points along the capillary length were plotted in FIG. 8b. The compound concentration is calculated, taking into consideration the original concentration of carbachol, the capillary length, expressed in mm and the concentration coefficient, C, calculated for each position of the capillary length. (calculated during the calibration run previously described in example 2). The data was plotted using Prism (GraphPad, USA) with a built-in Sigmoidal four parametric logistic function to obtain a compound LogEC50 values shown in Table of FIG. 8c. A plot of the Log EC50 values as a function of time FIG. 8c(B) reveals the potency and time-dependent behavior of the agonist in inducing a cell response. Here, it is evident that the potency of carbachol increases (Log EC50 decreases) until the 40 second mark, where it stabilizes. This dependence further allows for calculations of on-rate constants of the ligand interaction with the receptor.

### EXAMPLE 4

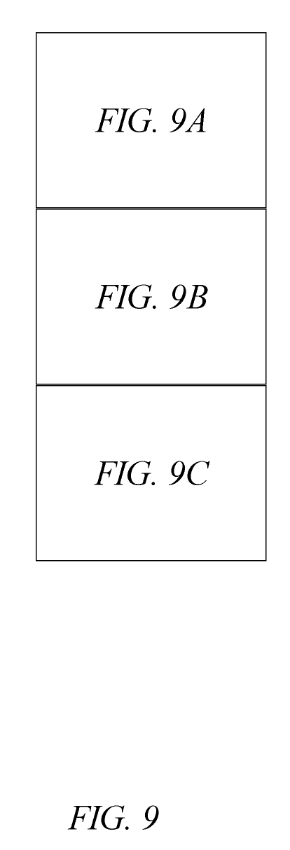

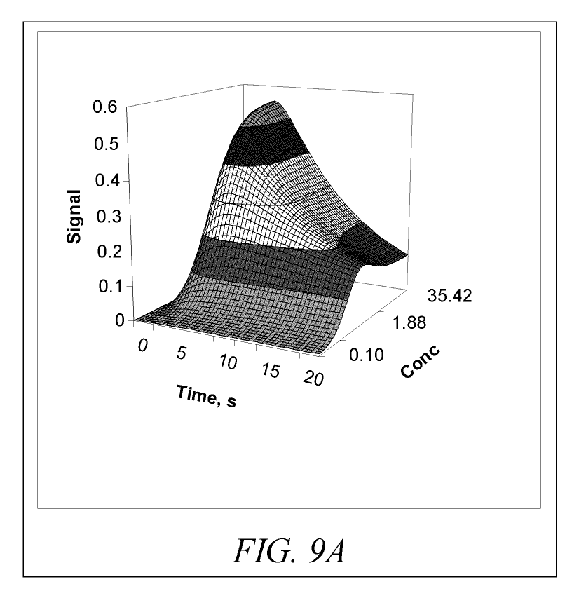

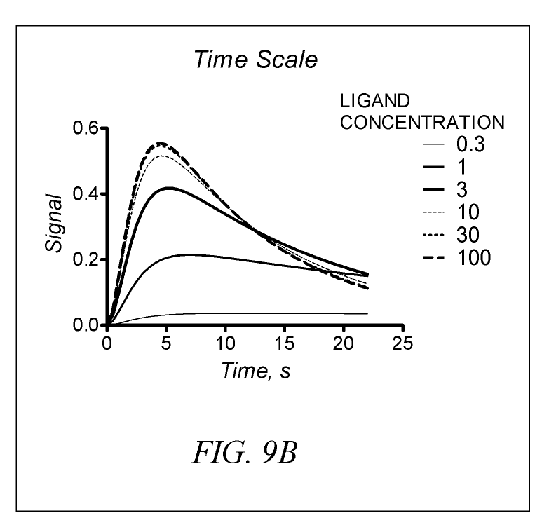

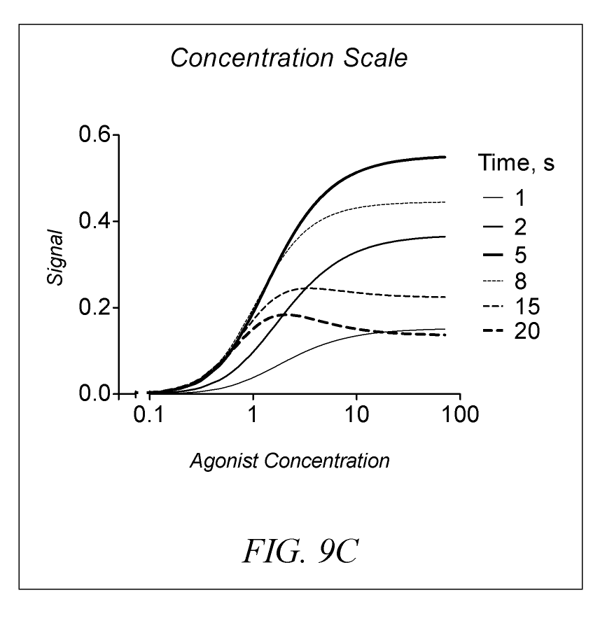

In 3-dimensional (3-D) mode, pump 110 picks up the agonist carbachol, (known agonist of m-cholinoceptors), as a test compound, from reservoir 106, into nozzle 116 (FIG. 1) and dispenses it into mixer 108. Simultaneously, pump 140 provides a dilution buffer from reservoir 148 to dilute the test compound in a variable speed fashion as described in Example 1, to create the test compound concentration gradient. Simultaneously, pump 130 provides a buffer (from the reagent reservoir 138) and pump 120 provides cells (from reservoir 128) into corresponding inlet ports of the mixer 108. The combined mixed flow of the four components, cells, agonist (at different concentrations), dilution buffer and reagent (in this case also buffer), fills in the capillary 150 and then stops (step 513, FIG. 5). Scanning head 184 performs repetitive measurements of the signal along the capillary length (a series of concentration curves consecutively registered in time). The resulting output of the signal as a function of both the test agonistic compound concentration and time of the signal evolution is shown in FIG. 9a. This data allows for analysis of both kinetic curves at selected concentrations (FIG. 9b) and concentration curves at selected time points (FIG. 9c). Using the Concentration Scale at different selected time points, one can calculate the potency and time dependence of the agonist (as shown in Example 3). Additionally, the Time Scale can be used to assess kinetics of the receptor activation at different agonist concentrations.

### EXAMPLE 5

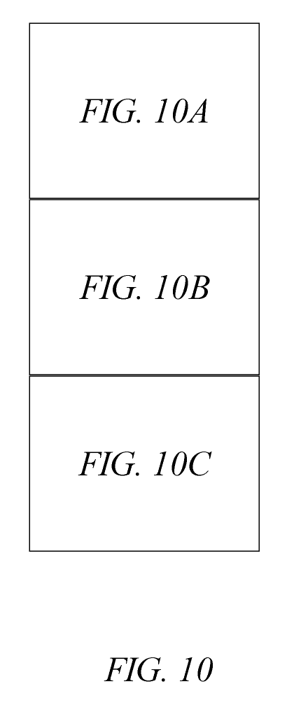

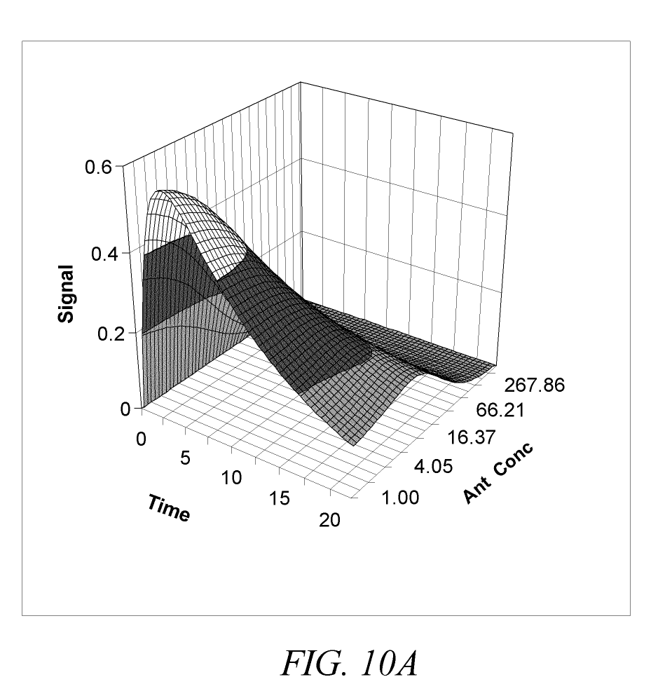

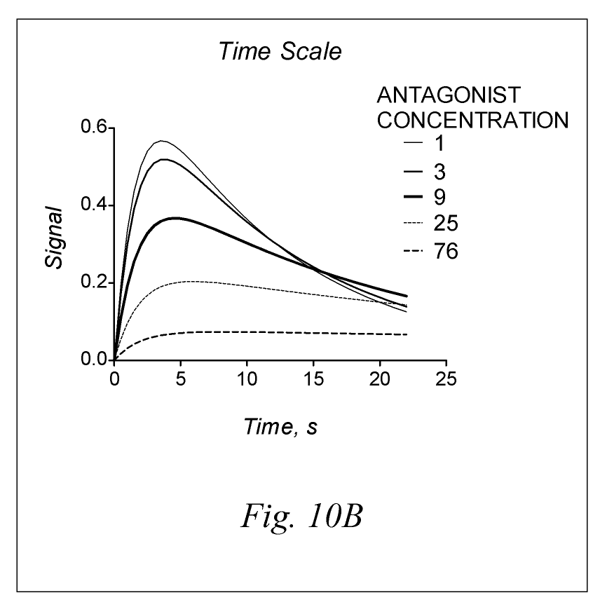

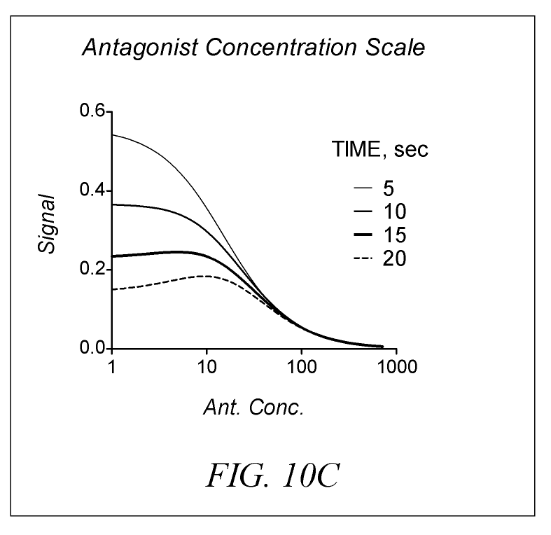

Pump 110 picks up a test compound, spiperon, (known antagonist of m-cholinoceptors) from reservoir 106, into nozzle 116 (FIG. 1) and dispenses it into mixer 108. Simultaneously, pump 140 provides a dilution buffer from reservoir 148 to dilute the test compound in a variable speed fashion as described in example 1, to create the test compound concentration gradient. Simultaneously, pump 130 provides an agonist, carbachol (from the reagent reservoir 138) and pump 120 provides RD cells loaded with Fluo 4 dye, as described in the Example 3, from reservoir 128 into the corresponding inlet ports of the mixer 108. The combined mixed flow of the four components (cells, antagonist/dilution buffer at different proportions, and agonist) fills in the capillary 150 and then stops (step 513, FIG. 5). Scanning head 184 performs repetitive measurements of the signal along the capillary length (a series of antagonist concentration curves consecutively registered in time). The resulting output signal, as a function of both the test agonistic compound concentration and time of the signal evolution, is shown in FIG. 10a.

Although the invention has been described in detail with reference to certain particular embodiments thereof, it will be understood that any variations and modifications apparent to those of skill in the art will still fall within the spirit and scope of the invention. Other embodiments not specifically described herein may fall within the spirit and scope of the present invention as provided by the following claims.

What is claimed is:

1. An apparatus comprising;
an automated test compound source capable of providing one or more test compounds at a programmably controlled flow rate;
an automated living cell or particle source capable of providing one or more cell or particle types at a programmably controlled flow rate;
an automated dilution reagent source capable of providing a dilution reagent at a programmably controlled flow rate;
a mixing chamber in fluid communication with said test sample source, said cell or particle source, and said dilution reagent source wherein said mixing chamber is adapted to combine a flow of at least one test compound received from said test compound source with a flow of dilution reagent received from said dilution reagent source, to provide a test solution, which is combined with the cells or particles received from said cell or particle source to generate a combined stream; and
a detector comprising:
i) a detection capillary liquidly coupled with the mixing chamber to receive and hold the length of the combined stream after the flow is stopped; and
ii) a detector, for measuring changes in properties of said cells, located along the length of the detection capillary.

2. The apparatus of claim 1, wherein said automated test compound source and said automated dilution reagent source are capable of creating variable flow rates of the test compound and the dilution reagent, respectively.

3. The apparatus of claim 2, wherein the flow rate of the test compound increases proportionally to the decrease in the flow rate of the dilution buffer to maintain a constant test solution flow rate while increasing the concentration of the test compound along a stream of the test solution.

4. The apparatus of claim 1, further comprising an automated standard reagent source capable of providing one or more standard reagents having a known effect on said cells to said mixing chamber at a programmably controlled flow rate, wherein said mixing chamber is adapted to combine a flow of said standard reagent with a flow of said dilution reagent to provide a standard solution that can be combined with cells or particles received from said cell or particle source to generate a combined mixture flow.

5. The apparatus of claim 4, wherein the flow rate of the standard reagent increases proportionally to the decrease in the flow rate of the dilution buffer to maintain a constant standard solution flow rate while increasing the concentration of the standard reagent along a stream of the reagent solution.

6. The apparatus of claim 4, wherein said combined mixture flow is comprised of the cells or particles, the test compound, the dilution buffer, and the standard reagent.

7. The apparatus of claim 1, further comprising a pump fluidly coupled with the exit end of the capillary that provides a pull out rate equal to rate of combined stream entering the capillary from the mixer under positive pressure created by combined flow rate of fluids into the mixer to eliminate a pressure drop during flow of fluids within the apparatus.

8. The apparatus of claim 1, further comprising a wash pump fluidly coupled with the exit end of the capillary to provide a counter-flow of at least one cleaning solution into said capillary and said mixing chamber and an outlet port on said mixing chamber coupled with a vacuum source to evacuate the cleaning solution.

9. The apparatus of claim 1, wherein said mixing chamber comprises:
a cell intake port fluidly coupled with said cell or particle source to receive stream of cells or particles from said test compound source;
at least one test sample intake port to receive said test sample from a nozzle fluidly coupled with said test sample source;
a dilution reagent intake port to receive dilution reagent from said dilution reagent source;
at least one mixing zone in fluid connection with the cell intake port, the test sample intake port, and the dilution reagent intake port;
an outlet port coupled with a vacuum source to evacuate a wash solution; and,
at least one outlet port liquidly coupled with said capillary to provide a combined stream to the capillary.

10. The apparatus of claim 9, further comprising a standard reagent intake port to receive standard reagent from the standard reagent source, which is fluidly coupled to the mixing zone.

11. The apparatus of claim 1, wherein said detector is selected from a photometer, a fluorometer, a cytometer, a radiometer.

12. The apparatus of claim 1, wherein said cell types or particle types are located in an array.

13. A method for determining a response of living cells to a test sample, comprising:
providing a flow of a test sample;
providing a flow of living cells;
combining said flow of test sample with said flow of living cells to form a combined flow of said test sample and said living cells in a mixing zone;
directing said combined flow of said test sample and said cells to a capillary;
stopping the combined flow when the capillary is filled in;
repeatedly detecting and measuring a response of the living cells to the test sample along the capillary length to affect a kinetic analysis of the effect of said test compound on said living cells.

14. The method of claim 13, wherein combining said flow of test sample with said flow of living cells comprises mixing said test sample and said living cells in a mixing zone.

15. The method of claim 13 further comprising:
providing an increasing flow of said test sample;
providing a decreasing flow of the dilution reagent;
providing a constant flow of said living cells;
combining said flow of test sample, dilution reagent and living cells to form a combined flow in said mixing zone;
directing said combined flow to a capillary;
stopping the combined flow when the capillary is filled in.

16. The method of claim 15, wherein a sum of flow rates of said test sample and said dilution reagent is constant, while the concentration of the test sample increases along the combined flow.

17. The method of claim 13 wherein kinetic responses from different locations in the capillary length represent independent traces from a multiplicity of cells located along the capillary at a constant concentration of the test sample.

18. The method of claim 13 wherein kinetic responses from different locations in the capillary length represent independent traces from a multiplicity of cells located along the capillary at variable concentrations of the test sample.

19. The method of claim 13, comprising determining a concentration-dependent response of said cells to said test sample after combining said test sample with said cells in said mixing zone.

20. The method of claim 13, wherein said response of said cells comprises a change in characteristics of said cells, induced by said test sample.

21. The method of claim 13, comprising determining a dose-time-response curve of said test sample on said living cells.

22. The method of claim 13, further comprising a retrowash wherein at least one wash solution is provided through output extremity of the capillary and vacuumed off through an outlet port in the mixing zone,

23. The method of claim 13 further comprising:
providing a flow of a standard reagent;
combining said flow of said standard reagent, with said flows of the test sample, the dilution reagent and the living cells to form a combined flow of said standard reagent, said test sample, said dilution reagent, and said living cells in said mixing zone;
directing said combined flow to said capillary;
stopping the combined flow when the capillary is filled in.

24. The method of claim 23 further comprising:
providing a constant flow of the standard reagent;
providing an increasing flow of said test sample;
providing a decreasing flow of a dilution reagent;
providing a constant flow of living cells;
combining said flow of said standard reagent, with the flow of the test sample, the dilution reagent and the living cells to form a combined flow in said mixing zone;
directing said combined flow to said capillary; and
stopping the combined flow when the capillary is filled in.

25. The method of claim 24, wherein a sum of flow rates of said test sample and said dilution reagent is constant, while the concentration of the test sample increases along the combined flow.

26. The method of claim 23, wherein kinetic responses from different locations in the capillary length represent independent traces from a multiplicity of cells located along the capillary at variable concentrations of the test sample in the presence of a constant concentration of the standard reagent.

27. The method of claim 23, comprising determining a concentration-dependent response of said cells to said test sample in the presence of constant concentration of said standard reagent after combining said test sample, said standard reagent and said cells in said mixing zone.

28. The method of claim 23, wherein said response of said cells comprises a change in characteristics of said cells, induced by said standard reagent in the presence of different concentrations of the test sample.

29. The method of claim 23, comprising determining a dose-time-response curve of said test sample on said living cells in the presence of said standard reagent.

30. The method of claim 23, wherein said test sample is selected from an agonist, an antagonist and an allosteric modulator.

31. The method of claim 23, wherein said standard reagent is selected from an agonist, an antagonist and an allosteric modulator.
